(19) United States
(12) Patent Application Publication
(10) Pub. No.: US 2012/0132593 A1
(43) Pub. Date: May 31, 2012

(54) SYSTEMS AND METHODS FOR MAGNETIC SEPARATION OF BIOLOGICAL MATERIALS

(75) Inventors: Sunil Srinivasa Murthy, Bangalore (IN); James William Bray, Niskayuna, NY (US); Shankar Chandrasekaran, Bangalore (IN); Arvind Kumar Tiwari, Bangalore (IN); Aaron Joseph Dulgar-Tulloch, Ballston Spa, NY (US); Munish Vishwas Inamdar, Bangalore (IN)

(73) Assignee: GENERAL ELECTRIC COMPANY, Schenectady, NY (US)

(21) Appl. No.: 12/957,240

(22) Filed: Nov. 30, 2010

(51) Int. Cl. B03C 1/02; B07C 5/34; C12M 1/42

(52) U.S. Cl. 210/695; 435/283.1; 210/222; 209/636

(57) ABSTRACT

A magnetic separator comprising a separation chamber is provided. The separation chamber comprises a having an inlet and at least one outlet opposite the inlet in a downstream direction, and a magnetic source operatively coupled to the separation chamber. The magnetic source comprises a plurality of magnets that can be selectively turned off and on to create a dynamic magnetic field in the separation chamber.

### BACKGROUND

The invention relates to systems and methods for separation of biological particles, and more particularly to systems and methods for magnetic separation of biological particles.

One of the key advances in cellular and molecular biology has been the development of separation techniques that are capable of identifying specific biological macromolecules. These separation techniques may be used for analytical and purification purposes in biological research, biomedical technology, and large-scale biochemical production.

Some of the separation techniques use one or more physical or chemical properties of biological macromolecules to modify their relative position. Properties that have been used to separate biological macromolecules include density, size, hydrophobicity, net charge, and specific surface chemical groups. The separation techniques that are commonly used in laboratories include centrifugation, liquid chromatography, and gel electrophoresis. In each of these techniques the position of the macromolecule of interest is modified in relationship to a moving phase or a stationary phase. For example, centrifugation may be used to separate cellular components based on their relative density if a stationary density profile is set up in the centrifuge tube. In liquid chromatography, a sample is passed over a packed column of particles that has a defined surface chemistry or porosity. This allows specific constituents to be retained in the chromatography column based on their surface chemistry or size. In gel electrophoresis, the relative charge-to-mass ratio of biological macromolecules is used to separate them in the presence of an applied electric field based their mobility through the gel in one or two dimensions. These separation techniques are widely used to measure the presence of a biological macromolecule and/or isolate a particular type of biological molecule from a complex mixture of macromolecules.

The separation technique selected to isolate a biological molecule is determined by the physical properties of the molecule of interest, the resolution of the separation to be performed, the scale at which the separation will be performed, and the availability of special reagents, such as antibodies, which make affinity separation possible. In general, biological separations need to be high resolution, which means that they are typically rather slow (e.g. most separation techniques take several hours) and are performed on relatively small volumes (e.g. most separation techniques are performed on 1-1000 ml volume samples).

While some separation techniques may involve separating particular biological material using non-immunological means, other separation techniques may use immunological means. The former approach has relied upon physical properties of the materials such as size, shape, density and charge. While this approach has yielded fast and simple isolation techniques they have lacked the desired specificity, especially in the case of cells. The latter approach, which involves attaching labels to the biological material using specific recognition factors like antibodies, receptors or receptor ligands, may provide a high degree of specificity but to date has not provided the desired throughputs with minimal damage to the materials being isolated. Fluorescent Activated Cell Sorting (FACS), a specialized type of flow cytometry, is able to isolate biological materials with minimal damage but it is limited in its throughput capacity. For instance, the typical bone marrow aspirate, which is a likely target of such separations, is about 1.5 L containing about 15×10⁶ nucleated cells/ml so that about 2.25×10¹⁰ nucleated cells need to be processed and the typical umbilical cord sample is about 100 ml containing about 5×10⁶ nucleated cells/ml so that about 5×10⁸ nucleated cells need to be processed. But FACS has a typical processing capacity of only about 50×10³ cells/second. Its use in such cell separations would lead to inordinately long separation times. To obtain practical separation times a sorting capacity of at least about 10⁶ cells/second is desirable. On the other hand, Magnetic Activated Cell Sorting (MACS) has a fairly high capacity but its batch procedure may result in damage to the material being separated. In addition, its batch procedure is labor intensive, not readily automated and in practice limited to binary sorting in which only a single target may be extracted from a sample.

Thus there is a need for a high throughput technique for separating a biological material, with minimal damage to the material being separated, high specificity and a sorting capacity of at least about 10⁶ units per second.

### BRIEF DESCRIPTION

In one embodiment, a magnetic separator is provided. The magnetic separator comprises a separation chamber having an inlet and at least one outlet opposite the inlet in a downstream direction, and a magnetic source operatively coupled to the separation chamber and comprising a plurality of magnets that can be selectively turned off and on to create a dynamic magnetic field in the separation chamber.

In another embodiment, a magnetic separation system is provided. The method comprises one or more magnetic separators. The magnetic separators comprise a separation chamber having an inlet and at least one outlet opposite the inlet in a downstream direction, a magnetic source operatively coupled to the separation chamber and comprising a plurality of magnets configured to be selectively turned on and off to provide a dynamic magnetic field in the separation chamber, a carrier fluid source operatively coupled to the magnetic separator, and configured to provide a continuous laminar flow of a carrier fluid in the magnetic separator, and a tagged biological material source operatively coupled to the magnetic separator for introducing two or more tagged biological materials in the magnetic separator, wherein the tagged biological materials comprise two or more biological materials tagged to corresponding one or more magnetically responsive particles.

In yet another embodiment, a method for magnetic separation of a biological material is provided. The method comprises providing a sample stream having one or more biological materials, wherein the biological materials are associated with one or more magnetically responsive particles, providing a dynamic magnetic field, and exposing the sample stream to the dynamic magnetic field to separate the one or more biological materials from the sample stream.

### DRAWINGS

These and other features, aspects, and advantages of the invention will become better understood when the following detailed description is read with reference to the accompanying drawings in which like characters represent like parts throughout the drawings, wherein:

FIG. 1 is cross-sectional view of an embodiment of a magnetic separator of the invention configured to provide a rotating dynamic magnetic field gradient;

FIG. 2 is a top view of the embodiment of the magnetic separator of FIG. 1;

FIGS. 3a-3o are graphs of examples of rotating magnetic field gradients produced using the plurality of magnets shown in FIG. 2;

FIG. 4a is a graph of an example of displacements of a tagged analyte tagged to a magnetically responsive particle that is 4 μm in diameter in a dynamic gradient corresponding to FIG. 3a;

FIG. 4b is a graph an example of displacements of a tagged analyte tagged to a magnetically responsive particle that is 4 μm in diameter a dynamic gradient corresponding to FIG. 3i;

FIG. 5 is an example of affects of multiple magnetically responsive particles attachment on motion of a tagged analyte in a rotating magnetic field gradient;

FIG. 6 is an example of affects on displacements of biological materials attached to magnetically responsive particles of different sizes;

FIG. 7 is a schematic drawing of an embodiment of a magnetic separator of the invention for providing a translational dynamic magnetic field;

FIG. 8 is a top view of the magnetic separator of FIG. 7;

FIG. 9 is a perspective view of an example of a stacked arrangement of magnetic separators for performing magnetic separation of two or more biological materials; and

FIG. 10 is a flow chart for the example steps for magnetically separation two or more tagged analytes.

### DETAILED DESCRIPTION

The systems and methods of the invention enable high throughput, high specificity separation of biological materials with enhanced resolution. One or more target biological materials may be separated from a sample comprising different types of biological and/or non-biological materials. In certain embodiments, a magnetic separator is provided for carrying out magnetic separation of tagged biological materials. The magnetic separator generally comprises a separation chamber having an inlet and at least one outlet opposite the inlet in a downstream direction, and a magnetic source operatively coupled to the separation chamber and comprising a plurality of magnets that can be selectively turned off and on to create a dynamic magnetic field in the separation chamber. In one embodiment, the separation of the biological materials may be a continuous process. In another embodiment, the separation may be a batch process. The terms “target biological material” and “tagged analyte” may be used interchangeably throughout the description. The target biological material that needs to be separated from the mixture may be tagged with one or more magnetically responsive particles to form a tagged biological analyte. The tagged analytes are then subjected to a dynamic magnetic field and sorted by size and/or magnetic content of the attached magnetically responsive particle.

One or more types of cells of a biological material may be separated from a much larger population of cells. To obtain separations in a reasonable time, the user may want to process a large amount of all the biological materials of a given type present in a given sample, including both those sought and those not desired, in a short time period. For instance, the typical bone aspirate sample used for the isolation of stem cells is 1.5 L and contains 2.25×10¹⁰ nucleated cells although only between about 0.01% and 0.1% of these cells are mesenchymal stem cells (MSC). Thus, it may be desirable to process the entire 2.25×10¹⁰ nucleated cells even though only a small proportion of them, between about 10⁶ and 10⁷, will be magnetically tagged and separated. In contrast, if the sample were adipose tissue the MSC content would be between about 1% and 10% and if the sample were umbilical cord blood and the target were T-cells the recovery could be as much as 10% of the white cells and if the target were granulocytes the recovery could be as much as 60%. Therefore the number of sample units may be substantially greater than the units of target material isolated. The process is operated so that at least about 10⁶ units of biological material of the given type per second are processed. In one example, 10⁷ units of a particular biological material are separated from a mixture comprising different types of biological materials.

The magnetic separation process generally uses a dynamic magnetic field having a high resolution. Also, the particle-particle interaction for the magnetically responsive particles is minimized because the particles are non-linearly separated in a dynamic magnetic field.

To more clearly and concisely describe the subject matter of the claimed invention, the following definitions are provided for specific terms, which are used in the following description and the appended claims. Throughout the specification, exemplification of specific terms should be considered as non-limiting examples.

The term “magnetically responsive particle” refers to any particle dispersible or suspendable in a carrier media without significant gravitational settling and separable from suspension by application of a magnetic field.

As used herein, the term “magnetic moment” refers to a tendency of a magnetic particle to align itself with a magnetic field.

The “critical frequency” refers to the switching frequency beyond which the particle cannot follow the traveling magnetic field anymore.

In certain embodiments, any biological material whose units can be tagged with magnetically responsive particles and then subjected to flow in a carrier medium may be separated. If the target biological material is contained in a cell it may be required to lyse the cell to release the biological material to facilitate a tagging reaction. The biological material may be configured to flow in defined units in a fluid medium. The biological material may comprise, but is not limited to, cells and biomolecules, such as proteins. In one embodiment, living cells that display a surface marker that may be used as a means of associating the cells with magnetically responsive particles, may also be separated using the systems and methods of the invention.

The carrier fluid may be any fluid that may transport the sample and the magnetically responsive particles in the desired concentrations and at the desired flow rates. It is convenient to minimize the viscosity of the carrier fluid so as to minimize the drag that the tagged biological material may experience when being deflected under the influence of the dynamic magnetic field. But the fluid must have sufficient viscosity to entrain the sample including the target biological material and the non-target biological material as well as the magnetically responsive particles in the laminar flow. Water is a suitable and inexpensive carrier fluid with a low viscosity. In some cases, the viscosity of water may be increased with appropriate thickeners such as sucrose to avoid settling problems, particularly if the separation chamber is fed from a reservoir. If it is known that the biological material may be adversely affected by exposure to pure water, a buffer may be added. For instance, if the target biological material is living cells, salt may be added to the aqueous fluid to render it isotonic to minimize cell rupture.

The magnetically responsive particles may be any particles of an appropriate size for association with target biological materials and for participation in a flow in a carrier medium and that are responsive to a dynamic magnetic field gradient. The magnetically responsive particles may be of varying sizes. For example, the particles may be small whereby a plurality of such particles may associate with a single unit of a target biological material. Alternatively, the magnetically responsive particles may be large whereby several units of the biological material may associate with one or more of the particles. In some embodiments, the magnetic separation may be minimally affected or not affected by the number of magnetically responsive particles coupled to a single cell of a biological material. For example, two magnetically responsive particles coupled to a cell type A and having a size of 2 microns will primarily behave as per their size and may not be interpreted as a single magnetically responsive particle of 4 microns. In other embodiments, the ratio of magnetically responsive particle to units of target biological material may be controlled so that the deflection of these units in a given magnetic field gradient is within a given range while in another embodiment it is enough that the units of the targeted biological material undergo some minimum deflection.

The magnetically responsive particles may be selected based on the magnetic content of the particles. A magnetic content of a magnetically responsive particle is a function of volume (size) of the magnetic particle and magnetic susceptibility of the magnetic particle. Magnetically responsive particles having different magnetic contents are tagged to different biological materials. In a dynamic magnetic field, the behavior of the magnetically responsive particles is largely determined by their magnetic contents. Hence, a magnetically responsive particle having a higher magnetic content behaves differently than a magnetically responsive particle having a relatively lower magnetic content. For example, the response time for a magnetically responsive particle having a higher magnetic content for a change in orientation of a magnetic field gradient may be faster as compared to a particle with a lower magnetic content. The magnetic susceptibility is a function of magnetic moment. The magnetic moment of the magnetically responsive particle may be used to separate the determined biological analyte. A magnetically responsive particle having a high magnetic moment or high magnetic susceptibility will be the fastest to align itself with the dynamic magnetic field.

The magnetically responsive particle may comprise a magnetic metal oxide core generally surrounded by an adsorptively or covalently bound sheath or coat bearing organic functionalities to which bioaffinity adsorbents may be covalently coupled. In one embodiment, polymer-coated paramagnetic microparticles may be used as magnetically responsive particles. The magnetically responsive particles may be coated with specific chemistries such that the particles have the ability to bind to the corresponding target analytes from a mixture of the biological materials. The magnetically responsive particles may be dispersed in the carrier media without rapid gravitational settling. In one embodiment, the magnetically responsive particles may include a metal oxide core surrounded by a stable silane coating to which a wide variety of organic and/or biological molecules may be coupled. Silanes are suitable coating materials for metal oxide cores by virtue of their silicon-functionalities and may be coupled to bioaffinity adsorbents through their organofunctionalities. The magnetically responsive particles may have a particle size between about 1 nanometer and 1000 microns. For example, particles between about 1 and 20 nanometers such as 16 nm super-paramagnetic iron oxide (SPIO) particles are suitable.

The magnetic characteristics of the magnetically responsive particles may range from having permanent magnetic moments to having inducible magnetic moments. The latter are more convenient because once the deflection is achieved and the particles pass out of the magnetic field they do not have a retained magnetic property that might induce agglomeration. In one embodiment, the magnetically tagged biological materials have magnetic moments/content in a range from about 0 to about 10 emu/g as determined by a magnetic field in a range from about 0.01 T to about 2 T.

The magnetically responsive particles may be associated with the units of the target biological material in any convenient manner which allows specific attachment to just the target biological material and results in a strong enough association to survive laminar flow and deflection in the separation chamber. Immunological interactions and ligand receptor interactions are suitable for this purpose. In the former case antibodies to the target biological material may be attached to the magnetically responsive particles while in the latter case a ligand to a receptor carried by the target biological material may be attached to the magnetically responsive particles. If the target biological material is an antibody or a receptor ligand the attachment approach can be reversed. In any case, the moiety used to associate the magnetically responsive particles with the target biological material may be directly or indirectly attached to the magnetically responsive particles. One suitable approach is to use magnetically responsive particles that are coated or otherwise functionalized with a member of a common binding pair, such as biotin or streptavadin, and antibodies or receptor ligands, that are bound to the other member of the pair.

The magnetically responsive particles may be superparamagnetic. Such particles typically do not become permanently magnetized after applying a magnetic field. The superparamagnetic nature of the magnetically responsive particles permits these particles to be re-dispersed without magnetic aggregate formation. Hence the particles may be reused or recycled. The stability of the silane coating and the covalent attachment of molecules thereto also affect the ability to reuse a given type of particle.

In one embodiment, it may be desirable to use larger magnetically responsive particles because they tend to deflect more easily. The magnetic force on a particle is generally dependent on its volume but the drag on the particles from the fluid medium when they move laterally in response to the magnetic field gradient is dependent upon their surface area. So, depending on the application, it may be preferable to have less surface area per unit volume. In one embodiment, the magnetic particles tagged to the biological material may be subjected to a rotating magnetic gradient. In another embodiment, the magnetic particles tagged to the biological material may be subjected to a translational magnetic field.

The dynamic magnetic field may be located within the separation chamber. In some embodiments, the dynamic magnetic field may comprise a rotating magnetic field that may have a rotating magnetic field gradient. The rotating magnetic field may rotate inside the separation chamber. The position of the north and south poles for the magnetic field may be varied, to move the center of the magnetic field from one position to another within the separation chamber. Along with the change in position of the rotating magnetic field, the strength of the magnetic field may be varied from one position to another inside the separation chamber. In one embodiment, the rotating magnetic field may have a modulating frequency. The rotating dynamic magnetic field may immobilize particles of relatively lower magnetization.

The rotating dynamic magnetic field may take various shapes including, but not limited to, circular, elliptical, square, or rectangular magnetic fields. The shape of the magnetic field may depend on the arrangement of the magnets about the periphery of the chamber.

In other embodiments, the dynamic magnetic field may be a translational magnetic field. The translational magnetic field (e.g. the magnetic field gradient) may travel inside the separation chamber along a given direction. The strength of the magnetic field may also vary at different positions along a given magnetic pathway. The period of time in which the magnetic field exists at a particular position may also vary.

In a dynamic magnetic field, the tagged analytes are separated by the magnetization-to-volume ratio of the attached magnetically responsive particles. A tagged analyte bound to a magnetically responsive particle having a first value of magnetic content, will move at a different rate than a tagged analyte that is bound to a magnetically responsive particle having a second value of magnetic content.

The plurality of magnets used for providing a dynamic magnetic field may comprise one or more types: permanent, permanent focusing, electromagnetic magnets, or magnetic or excitation coils. In one example, the permanent magnets may be used as a baseline magnetic field that is modulated using non-permanent magnets. In one example, the rotating magnetic field may be generated using three or more pairs of exciting coils. The coils may be arranged so that each pair of exciting coils is disposed diametrically opposite to the chamber. In one embodiment, an alternating current may be applied to the exciting coils thereby generating the rotating magnetic field. In the case of a translational magnetic field or alternating magnetic fields, two or more exciting coils may be arranged so that the fluid path inside the chamber is disposed between the exciting coils. In such cases, the fluid path is substantially perpendicular to the magnetic field gradient. In one embodiment, an alternating current is applied to these exciting coils thereby generating the alternating field perpendicular to the direction of fluid flow. The power supply for applying current to the magnetic coils may be controlled using an electrical switch.

In embodiments where the dynamic magnetic field is a rotating magnetic field, above certain frequency of rotation of the magnetic field, the onset of non-linearities in the transport behavior of the magnetically responsive particle may be evident. At a certain critical frequency, a specific population of magnetically responsive particles, having a magnetic content equal to or above a certain value, may no longer be able to change their alignment at the pace of the rotating dynamic magnetic, and may become stationary. These magnetically responsive particles may then be separated/isolated from the other particles. Subsequently, the frequency of modulation of the rotating field may be varied to be more than or equal to the critical frequency of the tagged analytes having a smaller or larger magnetic content or size. For example, in a sample having tagged analytes that are tagged to magnetically responsive particles of sizes 1 micron, 2 microns and 4 microns, first the tagged analyte having the magnetically responsive particle of 4 microns may be isolated by setting a modulation frequency above the critical frequency of the 4 micron particles. Subsequently, the tagged analyte having the magnetically responsive particle of 2 microns may be isolated by setting a modulation frequency above the critical frequency of the 2 microns particles.

In a dynamic magnetic field that varies translationally, the tagged analyte, having a magnetically responsive particle with higher magnetic content, moves faster than a tagged analyte bound to a magnetically responsive particle with a lower magnetic content. Accordingly, the tagged analyte with higher magnetic content will be displaced more than the tagged analyte with a lower magnetic content. The tagged analyte with a higher magnetic content may be the first to reach the outlet of the separation chamber and may be isolated from the other tagged analytes. Subsequently, the other analytes may be collected at the outlet at different times during the separation process. Generally, an outlet is “opposite” an inlet if the inlet and its opposite outlet maintain a laminar flow path between them when the carrier fluid laminar flow is initiated in the separation chamber. Thus an inlet and its opposite outlet are the upstream entry and downstream exit, respectively.

In certain embodiments, by using the frequency dependence, highly sensitive separation of magnetically responsive particles may be achieved based on fractional differences in diameter of the magnetically responsive particles. This separation may be achieved with high resolution based on the size and magnetic content of the magnetically responsive particles. An ability to tune the external driving frequency, to cause the migration velocities for different magnetically responsive particles to differ by several orders of magnitude, is feasible.

The separation chamber should be of a size and design to allow fluid flow at a rate sufficient to process at least about 10⁶ units preferably about 10⁷ units of biological material of a given type per second. The needed fluid flow rate depends on the concentration of the biological material in the stream being injected into the separation chamber, the flow rate of the injection stream and the overall volume of the separation chamber. The separation chamber may comprise a circular domain, a rectangular, or any other geometrical shape. One example of a separation chamber may be cylindrical with a length between about 50 mm and 200 mm, preferably between about 80 and 150 mm, a width of between about 20 and 100 mm, preferably between about 30 and 65 mm and a height between about 1 mm and 5 mm, preferably about 2 mm. In one example, the magnetic field gradient at any given point in the width of the separation chamber over a substantial portion of its length varies at a rate of about 0.1 T/cm.

In one embodiment the separation chamber is designed to separate two or more different magnetic particles. There may be two or more outlets. Each of the outlets may be positioned to receive different magnetic particles of different sizes.

The appropriate residence time in the separation region of the separation chamber of the biological material being subjected to separation is dependent on the time needed for deflection of the magnetically tagged biological materials to their assigned outlets. This is turn depends upon the deflection distances from the laminar flow path of the injected sample to the laminar flow paths which lead to the assigned outlets and the magnetic force experienced by the magnetically tagged target biological materials. This then depends upon the magnetic field gradient seen by the magnetically tagged target biological materials over their deflection path and the magnetic responsiveness of this tagged material. This responsiveness may be adjusted by altering the magnetic properties of the tagging particles or the ratio of these particles to the target biological materials. It is usually desirable to minimize the residence time to maximize the throughput of the separation process. Another approach extends the length of the separation zone. For any given flow rate of the input stream carrying the material to be separated, the residence time in the separation zone can be lengthened by increasing the length of the separation zone. In one embodiment, the residence times may be in excess of about 20 seconds, or in a range from about 30 seconds to about and 300 seconds, or from about 30 seconds to about 150 seconds. In one example, the residence time may be calculated using one or more of the size of the chamber, strength of the magnetic gradient, estimated magnetic content of a target analyte to generate sufficient time for the target analytes to achieve displacement from non-target analytes.

The separation zone of the separation chamber is generally the portion of the chamber that is subject to a magnetic field gradient effective to cause deflection of magnetically tagged target biological material. For instance, if the longitudinal edge of a separation chamber were placed in or adjacent to the air gap of a magnet but the chamber were longer than the air gap in that direction essentially only the portion of the chamber co-extensive with the air gap in that direction would be the separation zone unless some edge effects extended the useful magnetic field gradient a short distance. Thus, the residence time in the separation zone is the time available to cause the deflection that affects the separation.

The magnetic field gradient should be imposed on the separation chamber so that it causes the magnetically responsive particles to be deflected some distance out of their laminar flow pattern during the particles' residence time in separation zone of the separation chamber. Typically the magnetic field gradient is imposed at approximately a right angle to the direction of laminar flow. Such an arrangement maximizes the degree of deflection obtainable from a given magnetic field. It is desirable to have the magnetic flux decrease as a field progresses transversely across the separation chamber. This can be readily achieved by placing one edge of the separation chamber that is parallel to the direction of laminar flow between the poles of an appropriately designed permanent magnet or an electromagnet. The magnetic flux will then decrease as a field progresses towards the opposite edge. A convenient magnetic flux gradient in such an arrangement is between about 1 and 20 Tesla per meter (T/m). For example, a separation chamber that is about 55 mm wide and 2 mm high, and having a flux density at a pole of greater than about 1 T with the separation chamber centered in an air gap of about 25 mm, will yield a magnetic flux in the portion of the separation chamber between the poles of between about 0.3 T and 0.4 T, and will yield useful magnetic field gradients.

To ensure that some magnetically labeled target biological materials are not deflected too far, the magnetic field gradient end in the laminar flow path may be configured to lead to the assigned outlet for these materials. This can be accomplished, for example, by inserting the separation chamber into the air gap of the poles of the magnet so that the edge of the poles overlays the laminar flow path leading to the assigned outlet. The materials' deflection will cease when exposed to the uniform magnetic field between the poles.

Another approach is to adjust the process parameters so that each magnetically labeled target biological material is only deflected so that it is entrained in the laminar flow path leading to its assigned exit. The residence time and magnetic field gradient may be selected so that the deflection of any given magnetically labeled target biological material will not overshoot its intended laminar flow path.

The deflected tagged analytes may be more precisely focused to their intended outlets through the use of high permeability strips located adjacent these outlets. In one embodiment, a material with a permeability of about 500 or greater or a magnetized material may be used to focus the tagged analytes to the outlet. One approach is to use iron or nickel strips that are 1 mm wide by 20 mm long and 500 microns thick and oriented along their length in the direction of laminar flow and placed directly before an outlet.

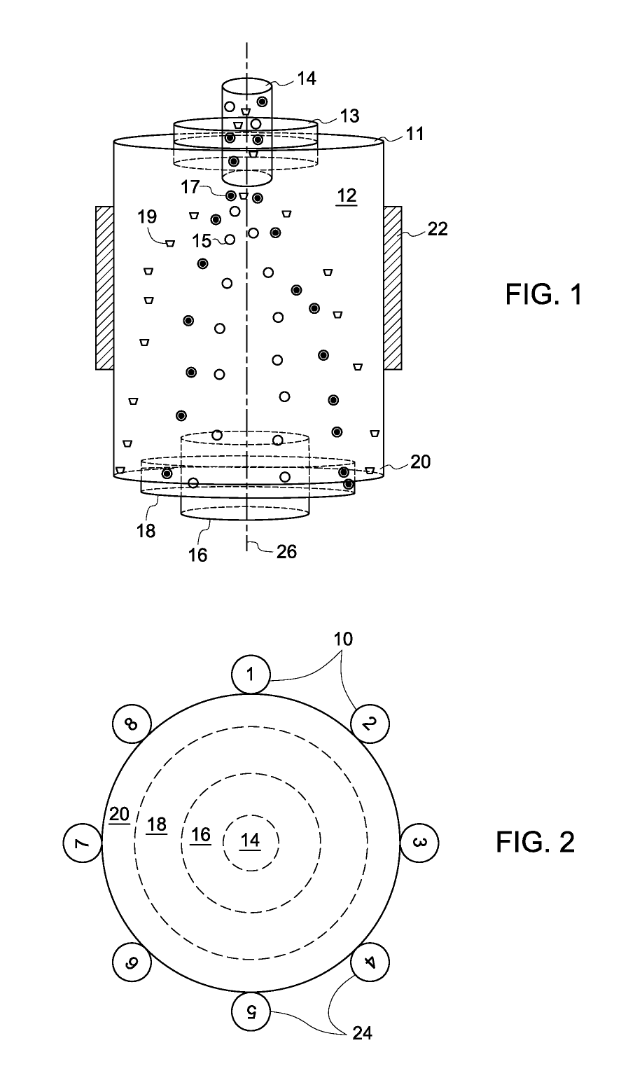

An embodiment of a magnetic separator of the invention is generally shown and referred to in FIGS. 1-2 as a magnetic separator 10. The magnetic separator 10 is configured to generate a rotational dynamic magnetic field. The magnetic separator 10 comprises a separation chamber 12. The separation chamber 12 comprises an inlet 14 for injecting the tagged biological material. Additional inputs 11 and 13 are provided to flow buffer solution to wet the flow path of the tagged analytes during operation while under influence of the magnetic field. In the illustrated embodiment, the tagged biological material comprises three different kinds of materials represented by reference numerals 15, 17 and 19. Outlets 16, 18 and 20 are used to collect the tagged biological material having magnetic particles of different sizes. The different outlets 16, 18 and 20 may be positioned depending on the properties of the magnetic responsive particles 15, 17 and 19.

A magnetic source 22 is disposed in operative association with the separation chamber 12. As illustrated in FIG. 2, the magnetic source 22 comprises a plurality of magnets 24. The plurality of magnets 24 is disposed around the periphery of the separation chamber 12. The plurality of magnets 24 may or may not be in physical contact with the separation chamber 14. The periphery about which the plurality of magnets 24 are disposed may be perpendicular to the central axis 26 of the separation chamber 12. The plurality of magnets (or magnetic poles) 24 may be made of electromagnets, permanent magnets, super-paramagnetic magnets, magnetic coils, or combinations thereof. In embodiments where all the magnets 24 are permanent magnets, the magnetic source 22 may be mechanically rotated about the chamber 12 to have a rotating magnetic field gradient. The different magnets in the plurality of magnets 24 may have the same or different magnetic strength. In one embodiment, a combination of permanent magnets and electromagnets is used. In this embodiment, the permanent magnets may be used to generate a base magnetic field to move the tagged biological material, and the electromagnets may be used to modulate the magnetic field gradient. In this embodiment, the modulation of the magnetic field gradient may be varied or controlled by selectively activating the electromagnets.

In one embodiment, the plurality of magnets 24 comprises electromagnets. The electromagnets may be of the same or different magnetic strengths. In one example, the plurality of magnets may conform to the surface of the separation chamber. In this embodiment, at least one surface of the magnets 24 may conform to the outer surface of the separation chamber 12. The magnets may or may not be in direct physical contact with the outer surface of the separation chamber 12. A dynamic magnetic field, having a rotating magnetic field gradient, may be generated by selectively activating two or more magnets at a time. In certain embodiments, the frequency of the rotating magnetic field gradient may be in a range from about 0.1 Hz to about 5 Hz. The frequency of the rotating magnetic field gradient may be determined based on the magnetic content of the magnetically responsive particles, the concentration of the tagged biological particles, or size of the separation chamber. In one embodiment, the frequency of the rotating magnetic field gradient may be selected based upon one or more of, the length of the separation chamber 12, the distribution of diameters of the magnetically responsive particles, or the number of magnetically responsive particles. In one embodiment, the frequency of the rotating magnetic field may be modulated. The response of the tagged analytes in a rotating dynamic magnetic field, having a modulating frequency enhances the resolution of the separation. In one embodiment, the frequency of the rotating magnetic field may be selected to make a magnetically responsive particle of a determined size stationary in the rotating magnetic field. The stationary magnetic particle may be collected closer to the central axis of the chamber 12.

By controlling the movement of the magnetic particles under the influence of the dynamic magnetic field at least about 20 percent variation in material properties may be tolerated. For example, the number of magnetically responsive particles attached to a particular biological material may be reduced or increased and yet still have no effect on the separation of the biological particles.

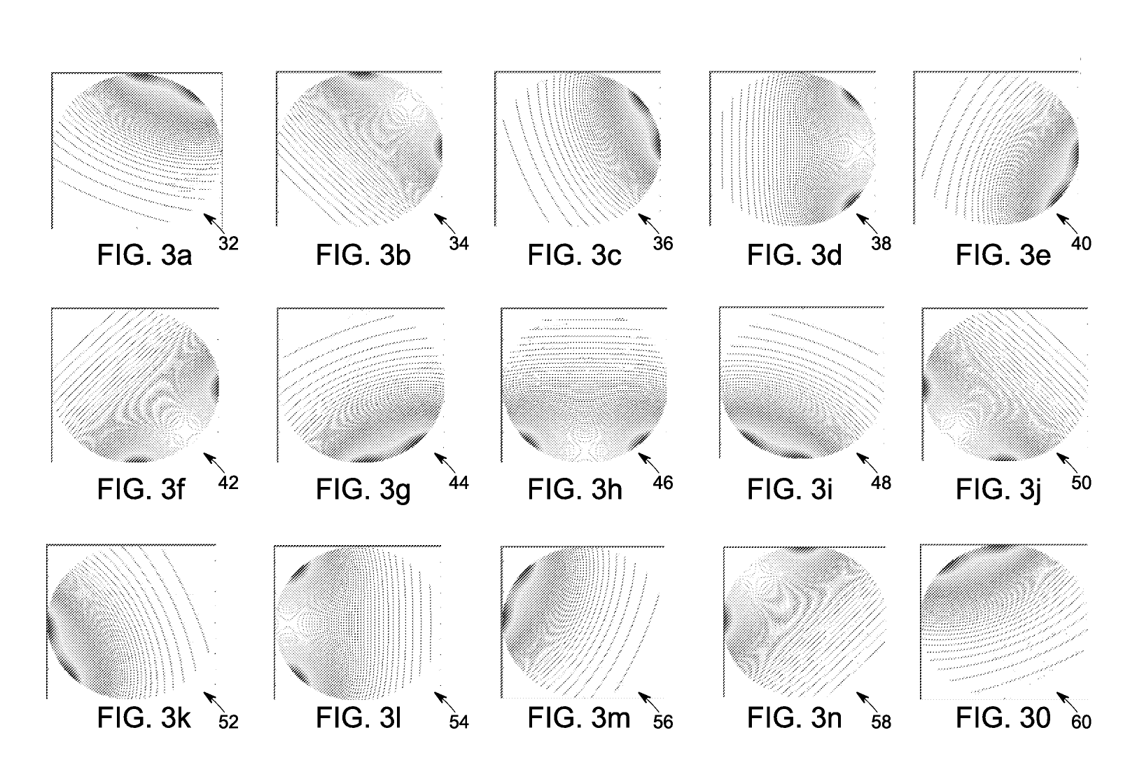

FIG. 3 illustrates an example of a rotating magnetic field gradient. In the illustrated embodiment, eight magnets are disposed about the periphery of the chamber 12 as illustrated in FIG. 2. Two magnets are excited at a given time, and the excitation of the magnet is continued for a small period of time in a range from about 0.5 seconds to about 7.5 seconds. The hold time is activation time during which each pair of coils is held active and the coil activation sequence is indexed to cover peripheral coils. The hold time or the time for which selected magnets are continued to be excited may depend on various factors such as velocity of flow of particles in the chamber, chamber length, or the magnetic content of the magnetic particles that are tagged to the biological material. In one example, the hold time may be cumulative for a constant circular frequency of the magnetic field. In one example, the hold time may be about 8 seconds, which would correspond to a frequency of rotation of the magnetic field gradient of about 0.125 Hz, and a switching time of about 0.25 seconds.

As shown in FIG. 3a, at first the magnets labeled 1 and 2 (see FIG. 2) are excited, and the excitation is continued for a period of about 0.5 seconds. One of the magnets acts as a north pole and the other as a south pole. The tagged biological material follows the path of the magnetic gradient 32. The particle with the higher content is the fastest to follow the magnetic field. Subsequently, as illustrated in FIG. 3b, as the excitation pattern is changed, and the magnets labeled 1 and 3 (see FIG. 2) are excited, the magnetic gradient changes to 34, and the excitation is continued for a period of about 1 second. The tagged biological material changes its trajectory and follows the new path of the magnetic gradient 34. The particle with the higher content is the fastest to follow the magnetic field, and the particle with the highest magnetic content also changes the path first and is the one that is deflected the most. After a period of 1 second, magnets 2 and 3 are excited, and the magnetic gradient changes to 36. The excitation is continued for a period of about 1.5 seconds. Likewise, the excitation patterns are changed as illustrated in Table 1.

| | Reference numeral | |
|---|---|---|
| Magnets excited | for corresponding | Hold time |
| (see FIG. 2) | excitation pattern | in seconds |
| 1 and 2 | 32 | 0.50 |
| 1 and 3 | 34 | 1.00 |
| 2 and 3 | 36 | 1.50 |
| 2 and 4 | 38 | 2.00 |
| 3 and 4 | 40 | 2.50 |
| 3 and 5 | 42 | 3.00 |
| 4 and 5 | 44 | 3.50 |
| 4 and 6 | 46 | 4.00 |
| 5 and 6 | 48 | 4.50 |
| 5 and 7 | 50 | 5.00 |
| 6 and 7 | 52 | 5.50 |
| 6 and 8 | 54 | 6.00 |
| 7 and 8 | 56 | 6.50 |
| 7 and 1 | 58 | 7.00 |
| 8 and 1 | 60 | 7.50 |

As the tagged analytes begin to shift under the influence of the rotating magnetic field gradient, the tagged analytes attempt to align themselves to the changing magnetic field gradient. The relative movements of the tagged analytes depend on the magnetic content of the magnetically responsive particles of the tagged analytes. The particles with different magnetic content follow different radii of curvature and may be collected at different distances from the central axis of the separation chamber. The magnetically responsive particles with higher magnetic content are influenced the most by the magnetic field. In one embodiment, at a certain frequency (also referred to as a critical frequency) of rotation of the magnetic gradient, the higher magnetic content particles are no longer able to follow the dynamic magnetic field and become stationary.

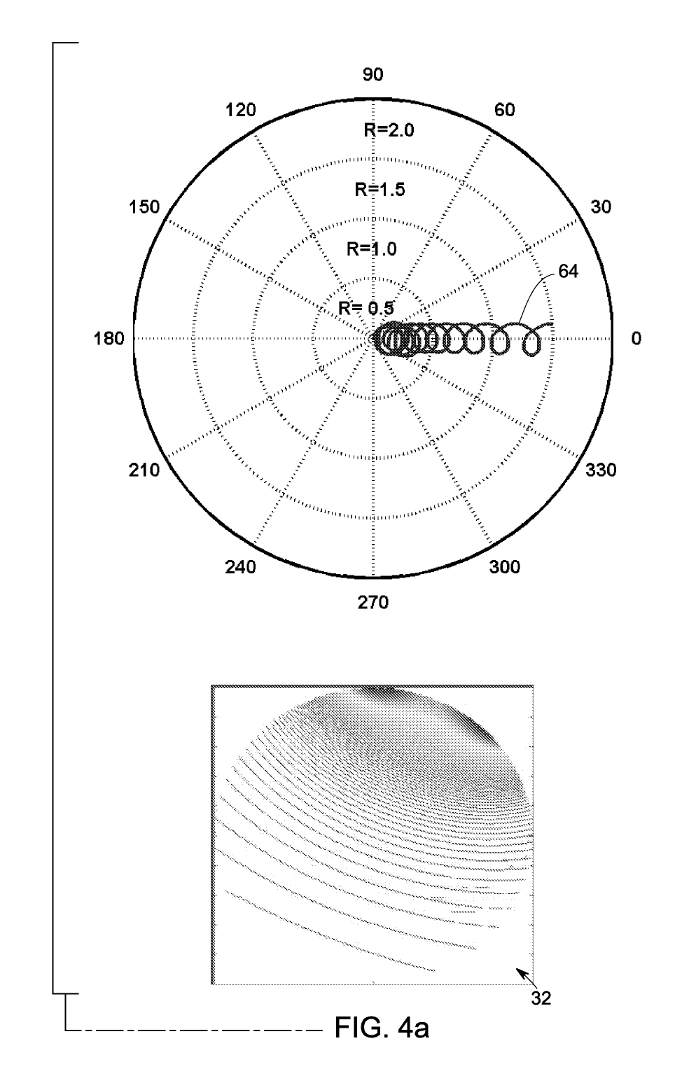

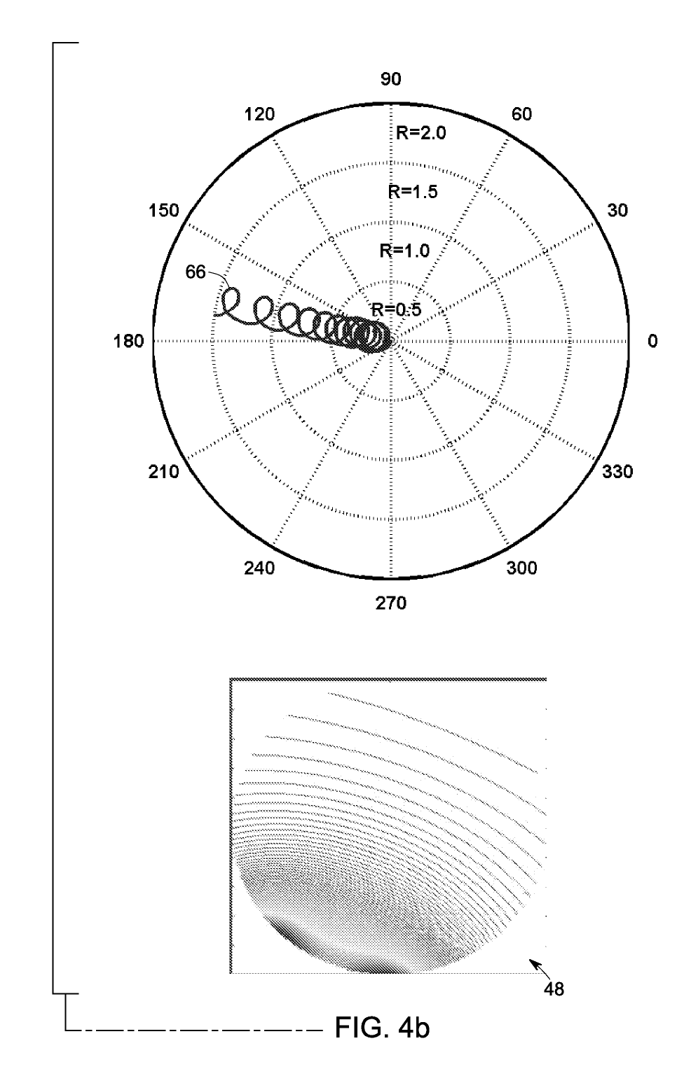

FIGS. 4a and 4b illustrate displacement of a tagged biological material that is tagged to one or more 4 microns size magnetic particles. As illustrated, the tagged biological material follows a spiral path 64. The spiral path 64 followed by the particle is under the influence of magnetic field of FIG. 3a where the magnets 1 and 2 are turned on for a period of 97.9 seconds. Similarly, the spiral path 66 followed by the tagged biological material is under the influence of magnetic field of FIG. 3i where the magnets 5 and 6 are turned on for a period of 98.6 seconds. The target analytes having same type of magnetically responsive particles respond to the magnetic field gradient in a similar fashion.

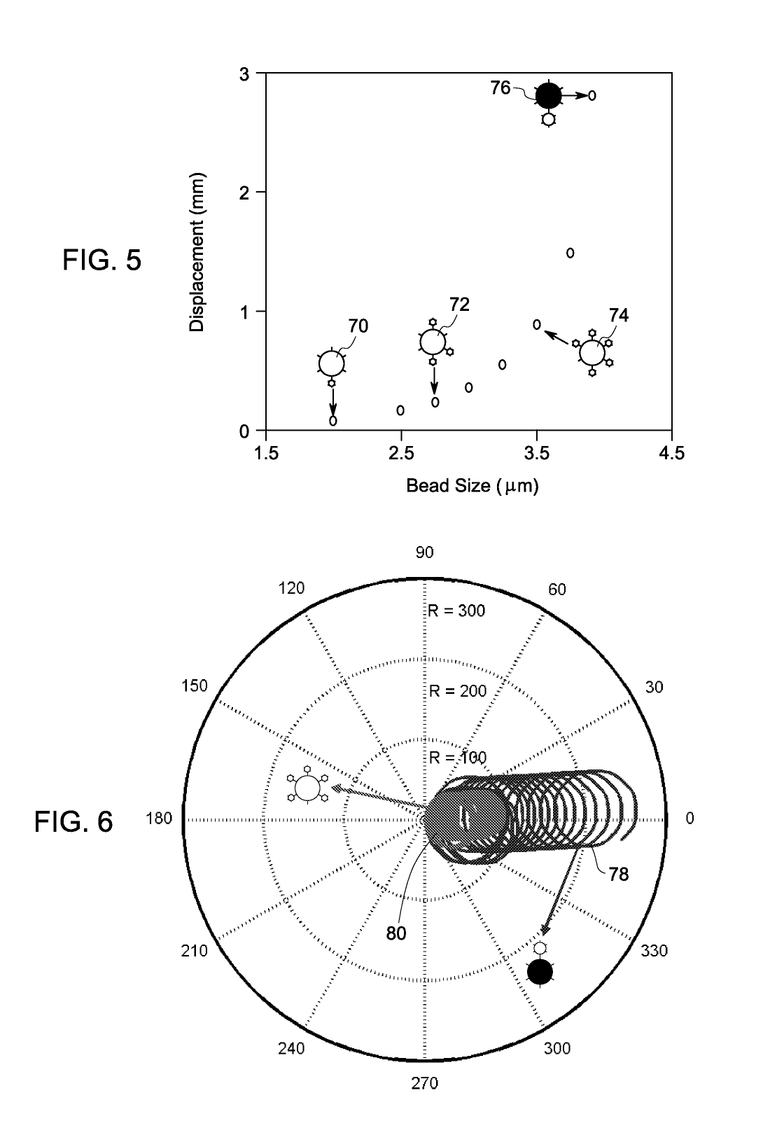

FIGS. 5-6 illustrate the reduced effect of multi bead (magnetically responsive particles) attachment to biological materials. FIG. 5 illustrates displacements (ordinate 68) of biological materials attached to 1, 3 and 5 magnetically responsive particles as represented by reference numerals 70, 72 and 74, respectively. The bead size for magnetically responsive particles 70, 72 and 74 is same. However, multiple beads attached to the cell have to be accounted for as illustrated in order to determine an “average” bead size. The abscissa 67 represents bead size while accounting for the number of beads attached to the biological particles 70, 72 and 74. The displacement of the tagged materials tagged to 1, 3 or 5 magnetically responsive particles is within a close range. In the illustrated embodiment, the magnetically responsive particles have a diameter of 2 microns. The displacement of a biological material coupled to a single magnetically responsive particle of 4 microns, as represented by a reference numeral 76, is very different from that of the biological materials attached to 1, 3 or 5 magnetically responsive particles having a diameter of 2 microns. This means that the system is tolerant to moderate differences in the number of magnetically responsive particles attached to biological material does not vary the displacement distance to any considerable degree. Hence, unlike existing magnetic separation techniques using constant magnetic field, the use of dynamic magnetic fields improves separation by making the separation process minimally affected by multiple number of magnetically responsive particles attaching to a biological particle. A biological material coupled to two 2 micron magnetically responsive particles will behave differently than a biological material coupled to a single four micron magnetically responsive particle. Also, displacements of biological materials coupled to 1 to 5 magnetically responsive particles, for example, may be within a close range.

FIG. 6 illustrates paths traveled by tagged analytes having biological material coupled to magnetically responsive particles of two different sizes. The magnetically responsive particles are coupled to similarly sized cells. The reference numeral 78 represents a spiral path traced by a target analyte having a biological material coupled to 5 magnetically responsive particles each having a size of about 2 microns. The reference numeral 80 represents a spiral path traced by a target analyte having a biological material coupled to a single magnetically responsive particle having a size of about 4 microns. As illustrated, the tagged analyte having a magnetically responsive particle with greater size or volume traverses the most distance regardless of the number of such particles coupled to the tagged analyte.

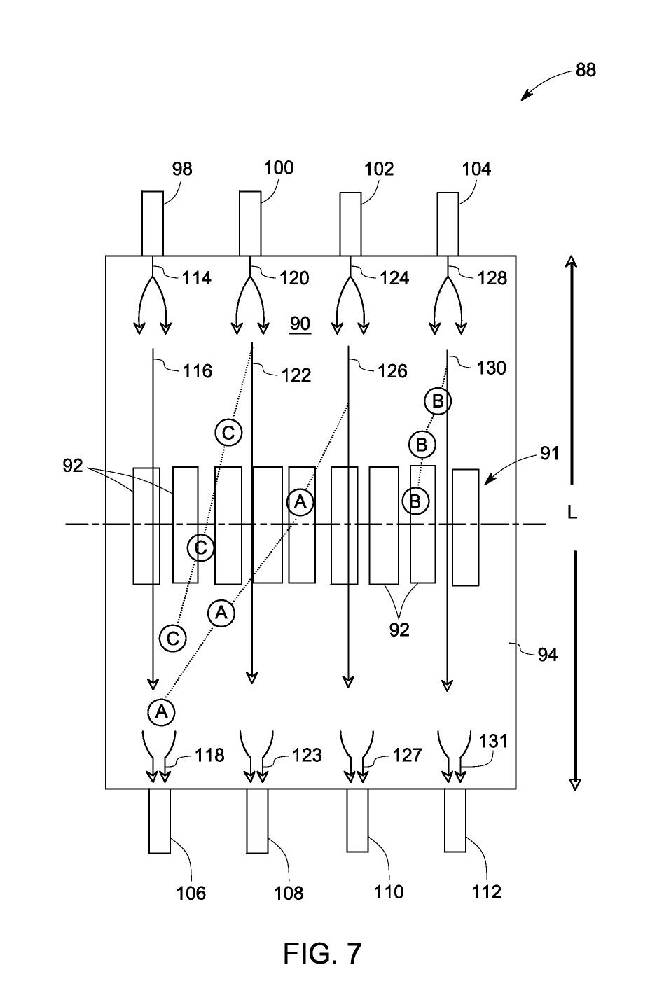

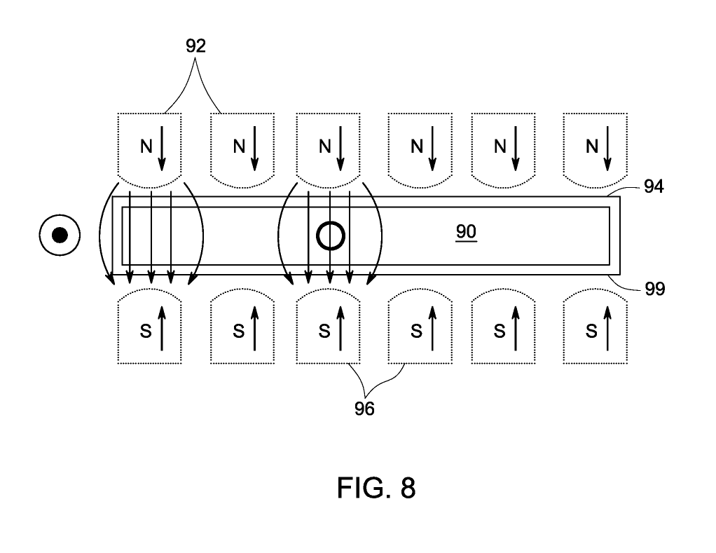

FIGS. 7-8 illustrate a magnetic separator 88 having a separation chamber 90 with a plurality of magnets 91. The magnets comprise a first set of magnets or magnetic poles 92 disposed on one side 94 of the chamber 90. A second set of magnets or magnetic poles 96 (see FIG. 8) are disposed on an opposite side 99 (see FIG. 8) of the separation chamber 90. Each of the magnetic poles 92 has a corresponding magnetic pole 96 disposed on the opposite side of the separation chamber 90. The magnetic poles 92 may be north poles and the magnetic poles 96 may be south poles, or vice versa. The magnetic poles 92 and 96 are disposed so that the direction of the magnetic field is perpendicular to the flow of the sample stream.

The poles 92 may conform to the surface of the separation chamber 90. In embodiments where the separation chamber is a rectangular structure, the poles 92 of the magnets 96 may be planar. The poles 92 are configured to provide a translational magnetic field along the length “L” of the chamber 90. The magnets 92 and 96 may have same magnetic strength. Alternatively, one or more of the magnets 92 and 96 may have magnetic strength, which is different from the magnetic strength of the other magnets.

Laminar flow of a carrier fluid is maintained by introducing the carrier fluid at inlets 98, 100, 102 and 104 and by withdrawing it from the opposite outlets 106, 108, 110 and 112, respectively. The beginning of the laminar flow path for inlet 98 is shown at 114, the center line at 116 and the end at 118. The laminar flow paths for inlets 100, 102 and 104 are similarly illustrated by 120, 122 and 123; 124, 126 and 127; and 128, 130 and 131, respectively.

The deflected distance traversed for a magnetically tagged target biological material A for an applied magnetic field at a given period of time may be different from the deflected distance traversed by magnetically tagged analyte B or C. In a dynamic magnetic field, the total distance traversed by the different analytes tagged to different magnetically responsive particles may be different. Hence, the different tagged analytes may reach the outlets at different times and at locations. For example, if the tagged analytes A are tagged to magnetically responsive particles with higher magnetic content, the tagged analyte A will be deflected

As this material is subject to the translational magnet field gradient imposed by the plurality of magnets 96, the tagged analyte A is deflected to a certain extent by a change in the dynamic magnetic field. The amount of deflection for the tagged analyte A is different from the amount of deflection of the tagged analyte B and C.

The separation chamber 90 should also have a length “L” in the direction of laminar flow to provide an adequate residence time for the units of the biological material being isolated to experience a deflection to an outlet or outlets not in the direct line of the modular flow. In a typical arrangement the chamber is provided with a sample inlet and several outlets with one of the outlets positioned directly opposite from the sample inlet such that sample entrained in the laminar flow of the fluid carrier will, in the absence of any force other than the laminar flow, pass from the inlet to this outlet. One or more other outlets are positioned so that the magnetically tagged target biological material (e.g. the biological material associated with magnetically responsive particles) may be deflected to them by a magnetic field gradient. The separation chamber 90 should be long enough so that practically imposable magnetic field gradients have sufficient time to cause the deflection. Deflection distances of greater than about 5 mm are convenient to obtain good separation while processing reasonable volumes of the biological material undergoing separation. As non-limiting examples, deflection distances between about 5 mm and 45 mm are preferred with distance of between about 10 and 30 mm being particularly preferred. Greater deflection distances in this range may be appropriate if more than one biological material is to be separated. For instance, if two different biological materials were to be separated simultaneously, one embodiment may assign the first offset outlet to the first material and the next offset outlet to the second material. Thus, the second biological material would require a different or greater deflection to reach its assigned outlet.

The magnetic field gradient can conveniently be given a suitable distribution across the width of the separation chamber by shaping of the magnetic poles imposing the magnet flux. For example, if the two poles are simply planar and parallel the gradient will drop sharply as one progress across the width of the separation chamber from the edge portion inserted between the poles to the opposite edge. This means that there will only be small differences in magnetic flux in adjacent portions near this far edge and consequently it will be more difficult to obtain the desired deflection of magnetically responsive particles in a suitable time, e.g. a residence time for particles suited to the fluid flow requirements. One approach is to use poles that are stepped or open in a V, wedge or curved shape with the mouth pointed to the far edge so that a magnetic field gradient will be created in the air gap of the primary magnet. This means a portion of the separation chamber that is inserted into this air gap can be used for separation instead of the uniform magnetic field that typically exists in the air gap of classic planar poles. Thus the distance over which an effective magnetic gradient is available to obtain deflection and thus separation is increased. In addition some pole shaping will moderate the drop off in magnetic flux in the region extending beyond the air gap thus extending the distance beyond the air gap in which there is still a sufficient magnet field gradient to affect deflection and thus separation. In one embodiment, smaller magnets may be disposed within the air gap of a larger magnet, e.g., the magnetic poles 92. This creates a magnetic field gradient within the air gap of the electromagnet. If a separation chamber is inserted into this air gap the portion of the chamber within this air gap would see a gradient that makes more of the separation chamber width available for separations. The effective separation zone of the separation chamber would not be limited to a short length extending from the edge of the poles until the field strength was so low as to no longer provide an effective gradient for deflection.

The laminar flow in the separation chamber may involve little if any turbulent flow or mixing. In one embodiment, the sample may enter the chamber at an inlet flow across to the outlet opposite the inlet in the direction of the laminar flow in the absence of any magnetic deflection. It certain embodiments, it may be desirable to avoid or reduce other lateral motion that could cause material, not subject to magnetic deflection, to exit a different outlet. Flow conditions including, but not limited to, the chamber design, the fluid velocity, the concentration of the biological materials of a given type and magnetically responsive particles in their laminar flow path, and the viscosity of the carrier fluid should conveniently be such that laminar flow is obtained.

It is convenient to have each of the inlets evenly spaced from the other inlets so that each laminar flow path is of approximately the same width as the other laminar flow paths. In such an arrangement, the average deflection distance for the target biological material and its associated magnetically responsive particle or particles will be approximately the same as the inlet spacing.

A further convenient feature of a separation chamber with multiple laminar flow paths is to have the laminar flow paths involved in the separation surrounded by uninvolved laminar flow paths. For instance, in a separation chamber with four inlets and four matched outlets, the two central outlets may be used for the separation while the outer two may support laminar flow paths which isolate the inner laminar flow paths from edge effects from the edges of the chamber. In such an arrangement, the sample containing the magnetically tagged target biological material would enter the separation chamber through one of the inner inlets and the magnetically tagged target biological material would be deflected into the laminar flow path originating from the other inner inlet.

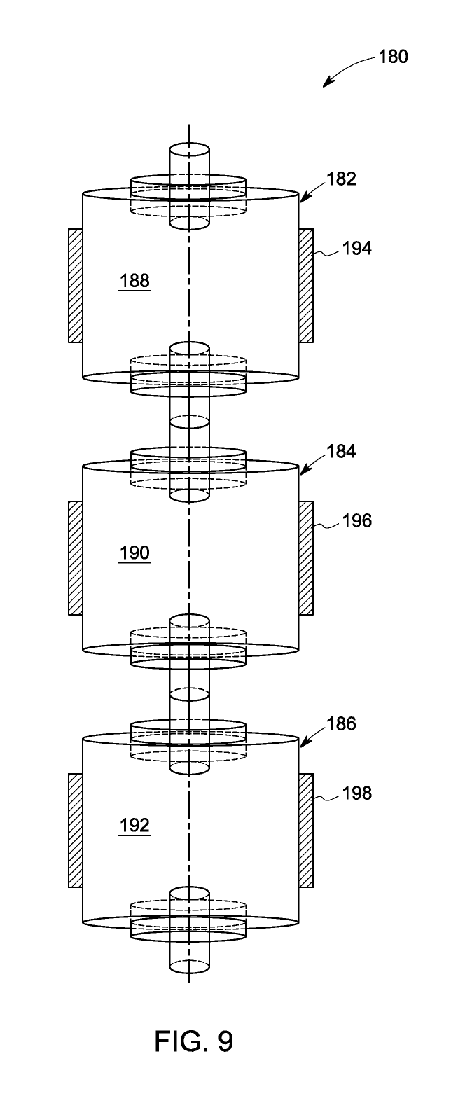

FIG. 9 illustrates an example where two or more magnetic separators are arranged so that an output from one magnetic separator may be used as an input for the next magnetic separator. In the illustrated embodiment, the arrangement 180 comprises three magnetic separators 182, 184 and 186. Any number of the magnetic separators may be used in the system 180. Also, although the arrangement 180 comprises magnetic separators are stacked, the magnetic separators may be arranged in any other fashion. For example, the magnetic separators may be disposed on a horizontal plane adjacent to another, and the output from one magnetic separator may be transferred to another magnetic separator using suitable transferring devices. The magnetic separators 182, 184 and 186 comprise separation chambers 188, 190 and 192, respectively and magnetic source 194, 196 and 198, respectively. The dynamic magnetic field in the different magnet separators 182, 184 and 186 may be varied differently. Also the strength of the plurality of magnets that are used in the different magnetic sources 194, 196 and 98 may vary from one magnetic separator to another. The combination of the magnetic separators 182, 184 and 186 may be used to magnetically separate mixture of biological material having a large number of different types of cells.

In one embodiment, some of the magnetic separators 182, 184 and 186 may be configured to have a rotating dynamic magnetic field, while the other magnetic separators may be configured to have a translating magnetic field.

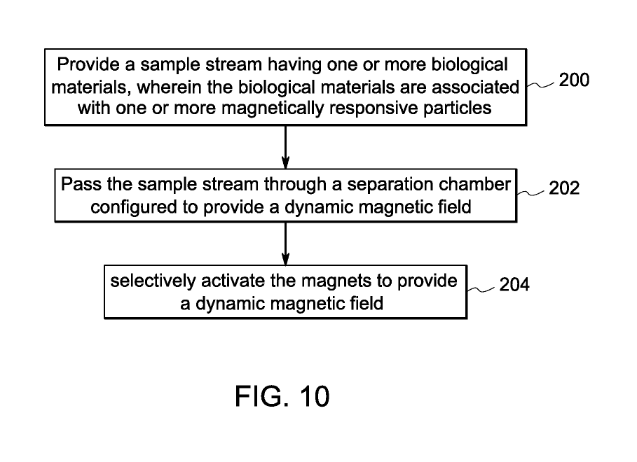

FIG. 10 provides a flow chart for magnetic separation using rotating magnetic field gradient. At block 200, a sample stream having one or more tagged biological materials is provided.

The sample comprising the target biological material is injected into an inlet of the separation chamber. The fluid flow rate at which this injection stream enters the separation chamber is important to the processing capacity of the process. The higher the fluid flow rate the greater the amount of biological material that can be processed per unit time. In one embodiment, the flow rates may be in a range from about 1 ml/min to about 5 ml/min.

The concentration in the injection stream of the biological materials to be subject to the separation process may be as high as possible without compromising the separation process. The higher the concentration of materials to be separated the more readily the throughput needed to obtain reasonable processing time is obtained. However, as the concentration of materials being subjected to magnetic deflection increases so does the probability of hydrodynamic effects that would cause the deflected material to entrain non-target biological material in its lateral motion. In addition, at higher concentrations the deflection may cause disturbance to the laminar flow and cause some stirring or mixing. In the case of cell separations, examples of total cell concentrations are between about 10⁷ cells/ml and 10¹⁰ cells/ml. [what is the basis for these ranges]. Similar concentrations are applicable to other types of biological materials such as biomolecules.

The injected biological material, other than that which is magnetically deflected, tends to remain in the laminar flow path between the inlet into which it is injected and the outlet opposite this inlet at the opposite end of the separation chamber. There is minimal dilution into the rest of the separation chamber. For example, this may be the case when the laminar flow path is sandwiched between two laminar flow paths of carrier fluid maintained between inlets on either side of the injection inlet and their respective outlets opposite these inlets at the opposite end of the separation chamber.

At block 204, the sample stream is passed through a separation chamber configured to provide a dynamic magnetic field to deflect biological material so as to cause it to exit the separation chamber from an outlet.

In one embodiment, where the dynamic magnetic field is a rotating magnetic field, a frequency of rotation of the dynamic magnetic field is varied so that some particles are stationary with respect to the others. The stationary particles may be collected from the outlet close to the central axis of the separation chamber.

In the case of a translational dynamic field, the injection inlet may be disposed between two carrier fluid inlets so that its laminar flow path is sandwiched between the laminar flow paths maintained between these inlets and their respective outlets opposite these inlets at the opposite end of the separation chamber. The one carrier fluid laminar flow path may serve to isolate the injection stream laminar flow path from any edge effects from a longitudinal edge of the separation chamber while the other carrier fluid laminar flow path can serve as the flow path into which the target biological material is deflected due to its association with magnetically responsive particles. In one embodiment, the second carrier fluid laminar flow path is isolated from edge effects by a third laminar flow path between it and the longitudinal edge of the separation chamber to which it is adjacent by an inlet and associated outlet between the inlet maintaining the deflection laminar flow path and this edge.

Isolating the laminar flow paths involved in the separation from edge effects may also be applied to multiple simultaneous separations. The laminar flow paths from the sample injection inlet and all the laminar flow paths leading to the outlets for the collection of the multiple target biological materials are collectively sandwiched between two laminar flow paths which run adjacent to the longitudinal edges of the separation chamber. Thus an inlet outlet pair is adjacent to each longitudinal edge to support a laminar flow path which is not involved in the separation process.

In certain embodiments, the method for separating the biological materials may be tolerant at least up to about 10 percent variation in the particle size. Sources of variation in commercially available magnetic particles include differences in size of the magnetic particle, variations in magnetic susceptibility and the randomness in the number of magnetic particles that may attach to a cell. In certain embodiments, the method and the system for separating the biological materials may be tolerant at least up to about 20 percent variation in the magnetic properties of the magnetic particles attached to the biological material. In some embodiments, the same kind of biological material, attached to different number of magnetically responsive particles, may be separated. The number of magnetically responsive particles may vary, for example, from 1 magnetic particle to about 5 particles. The tolerance to the number of magnetic particles may depend on the magnetization value, the size of the magnetic particles and strength of the magnetic field applied.

At block 202, some of the magnets are selectively activated, e.g., turned on and off, to provide a dynamic magnetic field. Magnetic separation is achieved by exposing the tagged analytes or magnetically labeled cells through the dynamic magnetic field. The magnetic field may travel or rotate from one location in the separation chamber to another. The different magnetic particles follow different paths and may be collected using a large annular area at the perimeter of the sorting domain. The rotating magnetic field may immobilize the tagged analytes of lower magnetization through control of the rotational frequency relative to the critical frequency of the smaller particles. These stationary particles essentially remain at the center of the sorting chamber and may be collected using a suitably (e.g. centrally) placed annular region. The magnetic force generated is sufficient to retain the target cells, while the unlabeled cells are washed away using a buffer solution. The targeted cell fraction is obtained by removing the column from the magnetic field and then washing out the previously attached cells.

In the case of a translational magnetic field, the biological particle tagged to the magnetically responsive particle having relatively higher magnetic content, may traverse the maximum distance at a given point of time. Also, the tagged analyte corresponding to this magnetically responsive particle may be the first one to traverse the length of the separation chamber and to exit at the opposite end of the separation chamber. In one embodiment, the different biological materials may be collected at different times in a separation chamber using translational dynamic magnetic field.

In certain embodiments, the methods and systems may be useful in a wide variety of analytical or clinical applications, such as but not limited to, separating macromolecules, e.g., DNA, RNA, polypeptides, proteins, and antibodies, as well as cells, e.g., stem cells, erythrocytes and white blood cells, and pathogens, e.g., viruses, bacteria, fungal spores. For example, the methods and apparatus may be used for isolating stem cells from bone aspirate or umbilical cord blood. Advantageously, the methods are gentle on cells and hence result in very limited cell damage during the separation process. The methods and systems provide an option to sort multiple cell types in a single flow through process that is fully contained and can be easily automated.

While only certain features of the invention have been illustrated and described herein, many modifications and changes will occur to those skilled in the art. It is, therefore, to be understood that the appended claims are intended to cover all such modifications and changes as fall within the scope of the invention.

1. A magnetic separator, comprising:
a separation chamber having an inlet and at least one outlet opposite the inlet in a downstream direction; and
a magnetic source operatively coupled to the separation chamber and comprising a plurality of magnets that can be selectively turned off and on to create a dynamic magnetic field in the separation chamber.

2. The magnetic separator of claim 1, wherein the plurality of magnets are disposed along a periphery of the separator unit, wherein the plurality of magnets are so arranged to provide a rotating dynamic magnetic field.

3. The magnetic separator of claim 1, wherein a frequency of modulation of the dynamic magnetic field is in a range from about 1 Hz to about . . . 1000 Hz.

4. The magnetic separator of claim 1, wherein the separation chamber comprises a cylinder, a cube, or a cuboid.

5. The magnetic separator of claim 1, wherein a length of the separation chamber is sufficient to provide a tagged target biological material an adequate residence time to be deflected by the dynamic magnetic field.

6. The magnetic separator of claim 1, wherein a material of high magnetic permeability or a magnetized material is placed adjacent to the outlet adapted to receive a tagged target biological material so as to focus the tagged biological material to the outlet.

7. The magnetic separator of claim 1, wherein the separation chamber is configured to allow a continuous flow of a carrier fluid carrying one or more tagged biological materials.

8. The magnetic separator of claim 1, wherein the plurality of magnets are conformal with an outer surface of the separation chamber.

9. The magnetic separator of claim 1, wherein the plurality of magnets comprise a permanent magnet, an electromagnetic magnet, a permanent focusing magnet, an electromagnet coil, or combinations thereof.

10. The magnetic separator of claim 1, wherein the magnetic system separates biological material, wherein the biological material comprises a living cell.

11. The magnetic separator of claim 10, wherein the biological material comprises a protein or nucleic acid.

12. The magnetic separator of claim 1, wherein a concentration of the biological material is greater than about 10⁷ units/ml.

13. The magnetic separator of claim 1, wherein a flow rate of the injection stream into the inlet is greater than about 1 ml/min.

14. A magnetic separation system, comprising:
one or more magnetic separators, comprising:
a separation chamber having an inlet and at least one outlet opposite the inlet in a downstream direction;
a magnetic source operatively coupled to the separation chamber and comprising a plurality of magnets configured to be selectively turned on and off to provide a dynamic magnetic field in the separation chamber;
a carrier fluid source operatively coupled to the magnetic separator, and configured to provide a continuous laminar flow of a carrier fluid in the magnetic separator;
a tagged biological material source operatively coupled to the magnetic separator for introducing two or more tagged biological materials in the magnetic separator, wherein the tagged biological materials comprise two or more biological materials tagged to corresponding one or more magnetically responsive particles.

15. The magnetic separation system of claim 14, comprising two or magnetic separators in fluid communication with each other, wherein an output of a magnetic separator is an input for a subsequent magnetic separator.

16. The magnetic separation system of claim 15, wherein a magnetic field strength of at least one of the magnetic separators is different from others.

17. The magnetic separation system of claim 15, wherein the magnetic separators are configured to provide a rotating magnetic field, or a translating magnetic field.

18. The magnetic separation system of claim 14, further comprising a tagged biological material separator unit for separating the magnetically responsive particles from the biological materials.

19. The magnetic separation system of claim 14, further comprising a cleavage unit for enzymatic cleavage of linker from the biological particles.

20. A method for magnetic separation of a biological material, comprising:
providing a sample stream having one or more biological materials, wherein the biological materials are associated with one or more magnetically responsive particles;
providing a dynamic magnetic field; and
exposing the sample stream to the dynamic magnetic field to separate the one or more biological materials from the sample stream.

21. The method of claim 23, comprising flowing the sample stream through a separation chamber configured to provide a dynamic magnetic field or magnetically separating the biological materials.

22. The method of claim 21, wherein a flow of the sample stream is a continuous flow.

23. The method of claim 20, comprising residing the sample stream in a separation chamber for a determined period of time for magnetically separating the biological materials.

24. The method of claim 20, wherein providing the dynamic magnetic field comprises:
providing a plurality of magnets operatively coupled to a separation chamber; and
selectively activating two or more of the plurality of magnets in a determined order, wherein the plurality of magnets are activated for a determined period of time

25. The method of claim 23, wherein providing the dynamic magnetic field comprises providing a rotating magnetic field.

26. The method of claim 25, comprising modulating a frequency of the dynamic magnetic field.

27. The method of claim 26, comprising modulating the frequency of the dynamic magnetic field so that at least one biological particle is stationary with respect to other biological particles.
